(19) United States
(12) Patent Application Publication
(10) Pub. No.: US 2019/0201666 A1
(43) Pub. Date: Jul. 4, 2019

(54) Universal retainer for surgical drains and tubing

(71) Applicant: Joanie Antholz, Duncan, OK (US)

(72) Inventor: Joanie Antholz, Duncan, OK (US)

(21) Appl. No.: 16/501,181

(22) Filed: Mar. 2, 2019

Related U.S. Application Data
(63) Continuation-in-part of application No. 13/462,806, filed on May 2, 2012, now abandoned.

(51) Int. Cl. A61M 25/02; A61M 27/00; A61M 39/08

(52) U.S. Cl. CPC A61M 25/02; A61M 27/00; A61M 39/08; A61M 2209/088; A61M 2025/024; A61M 2025/026; A61M 2025/0266; A61M 2025/0206

(57) ABSTRACT

A universal application drain bulb and tubing support system to secure one or more drain bulbs and its tubing subsequent to a surgical procedure, providing for multiple location placement of drain bulbs and tubing on a patient to reduce the incident of damage to the drain bulbs and tubing, early removal, risk of injury, infection or displacement and other common risks and hazards commonly incurred with drain bulbs and tubing.

### CROSS REFERENCE TO RELATED APPLICATIONS

This is a Continuation-in-Part application of U.S. patent application Ser. No. 13/462,806, filed on May 2, 2012 by the same inventor.

### I. BACKGROUND OF THE INVENTION

### 1. Field of Invention

A universal application drain bulb and tubing support system to secure one or more drain bulbs and its tubing subsequent to a surgical procedure, providing for multiple location placement of drain bulbs and tubing on a patient to reduce the incident of damage to the drain bulbs and tubing, early removal, risk of injury, infection or displacement and other common risks and hazards commonly incurred with drain bulbs and tubing.

### 2. Description of Prior Art

A preliminary review of prior art patents was conducted by the applicant which reveal prior art patents in a similar field or having similar use. However, the prior art inventions do not disclose the same or similar elements as the present drain bulb and tubing support system, nor do they present the material components in a manner contemplated or anticipated in the prior art.

A number of prior art patents involve pouches or bags which are intended to retain and secure drain bulbs used in post-surgical care. In the parent application to the present drain bulb support system, U.S. Patent Publication No. 2013/0296814 to Antholz, a belt is disclosed attaching a pouch which holds a drain bulb with a hook and loop strap holding the drain tube between the surgical site and the drain bulb close to the sub-pouch. The pouch is attached to the patient by a strap or an adhesive. In U.S. Patent Publication No. 2011/0230863 to Lentini, a garment and a belt with a plurality of secure pockets with a small aperture to provide for passage of the tubing, allows for the placement and storage of multiple bodily fluid drainage reservoirs around the waist. Several other design patents indicate belts with at least one pouch to accept surgical drain tubes, including U.S. Patents No. D437,410 and D 488,227, both to Watson. Two prior art patents define belts that use either a snap loop or hook and look fastener to secure a post-surgical drainage device, as disclosed in U.S. Pat. No. 6,273,872 to Friedman and U.S. Pat. No. 6,610,032 to Prody.

Related devices indicate a storage pocket which is worn by a patient for securing peritoneal dialysis tubes retaining a connection in a sterile pouch for the catheter to fill-drain tube, U.S. Pat. No. 6,682,507 to Irish, a body attachable bag as shown in U.S. patent No D311,453 to Rice, and a secure belt worn pouch as disclosed in U.S. Pat. No 6,925,691 to Bristel.

The prior art does not disclose the drainage bulb and tubing securing system of the present invention as disclosed and claimed which define a universal base panel defining a plurality of formed slots for securing one or more belts, bands or strap defining at least one secure pocket conforming to a surgical drain bulb with the base panel further providing a drain tubing anchor above the at least one secure pocket which maintains the post-surgical drain tubing against the base panel and further maintains an upright position of the drain bulb in a respective secure pocket, with the base panel further providing perforations between each secure pocket in those embodiment having more than one secure pocket, for separation of the pockets for multiple patients requiring multiple drain bulbs at one time, each secure pocket further defining a distinctive indicator to identify the drain bulb and corresponding site of each contained drain bulb and attached drain tubing.

### II. SUMMARY OF THE INVENTION

Post-surgical drain tubing and drainage bulbs are commonly used to drain and withdraw fluids from a patient where fluid retention and drainage of these post-surgical fluids is indicated. These tubes are installed within the patient at a location where the surgery has occurred and post-surgical fluid build up is predicted. Withdrawal of the fluids is critical and the most common means of draining these fluids is performed by the tubing which extends from the internal surgical site to a drain bulb. These drain bulbs, especially those manufactured by Jackson Pratt Bulb Drains, apply a suction upon the drain tubing and also a reservoir for the withdrawn fluids which are periodically compressed to create the appropriate suction force and periodically detached to dispose of the withdrawn fluids. A secure drain tubing and drainage bulb means is provided to secure the drain tubing and drainage bulbs to a patient for preventative purposes as well as to provide for a more cautious patient mobility risk reduction.

When the drain tubing and drainage bulbs are not secured properly, the tubing and bulbs may become damaged during patient movement or destabilized by incidental contact during patient activity. This is a significant cause of infections and destabilization of the post-surgical apparatus that stem from early drain removal, resulting is subsequent surgeries to reinstall the tubing, repair torn suture sites, or alleviate internal damage and infection. Proper bulb and tubing stabilization also results in patient safety and comfort. By providing the present drain bulb and tubing support system in a universal product, as disclosed within the drawings and claimed subject matter, health care providers can also provide for proper placement of the one or more corresponding drain tubing and drainage bulbs at multiple locations in proximity to each corresponding surgical site and also use multiple means of attachment to secure each respective system component pocket to the patient for ready observation of each respective segment of drain tubing and corresponding drainage bulb, monitor the healing of each surgical site and provide the patient with less restricted mobility than provided for in prior art devices.

### III. DESCRIPTION OF THE DRAWINGS

The following drawings are submitted with this utility patent application.

FIG. 1 is a perspective front view of the universal application drain bulb and tubing support system with pockets containing respective drain tube bulbs and drain tubing securing to the front surface of the flat base structure by respective drain tubing anchors.

FIG. 2 is a rear view of the flat base structure indicating an adhesive applied to the rear surface with a peel-off backing pre-applied over the adhesive.

FIG. 3 is an upper front perspective view of a pocket indicating the presence of a drain tube bulb within the pocket.

FIG. 4 is a perspective view of a first embodiment of a tubing anchor.

FIG. 5 is a top view of the first embodiment tubing anchor.

FIG. 6 is a perspective view of a second embodiment of a tubing anchor.

FIG. 7 is a top view of the second embodiment tubing anchor.

FIG. 8 is a front perspective of a tubing anchor indicated a hook and loop fastening strap.

FIG. 9 is an indication of a variety of placement locations for the universal application drain bulb and tubing support system.

### IV. DESCRIPTION OF THE PREFERRED EMBODIMENT

Many surgical procedures utilize post-surgical drain tubing X and drain bulbs Z to externally evacuate fluids which accumulate internally subsequent to a surgical procedure. Surgeries commonly associated with the use of drain tubing and drain bulbs include abdominal and chest cavity surgeries, organ transplants, neck, hip, prostate, thyroid, gall bladder, mastectomies, plastic surgery, breast implant and reductions, brain surgeries and many other medical invasive surgical procedures involving removal or addition of components of the human body. The drain tubing X is installed during the surgical procedure to drain collected fluids within the body with the intent to later remove the drain tubing X once the potential of fluid accumulation at the surgical site has resolved, at which time the drain tubing X is removed. To promote and accommodate the withdrawal of post-surgical fluids, the drain bulb Z is attached to the outer end of the drain tubing X to provide a negative pressure on the drain tubing X to withdraw the fluids from the surgical site inside the patient and also to provide a reservoir for the withdrawn fluids which is periodically disposed of during the post-surgical care.

As previously indicated, the period of time that these drain tubing X is surgically installed within the patient, a most critical stabilization of the drain tubing X and drain bulb Z is essential. The drain tubing X generally extend through the skin through either a surgically sutured exit site or a temporary port. The drain tubing X is placed within a void or cavity post-surgery to drain accumulating fluids which require periodic draining and forced evacuation. These ports or sutured exit sites are very delicate, fragile and susceptible to tearing, tubing dislocation and movement which can provide a pathway for infection, bleeding wound or surrounding tissue damage. Stabilization of the drain tubing X and drain bulbs Z encourages quicker and more efficient healing, decreases medical cost, reduces the chance of subsequent medical intervention, and prevents post-surgical disease and infection.

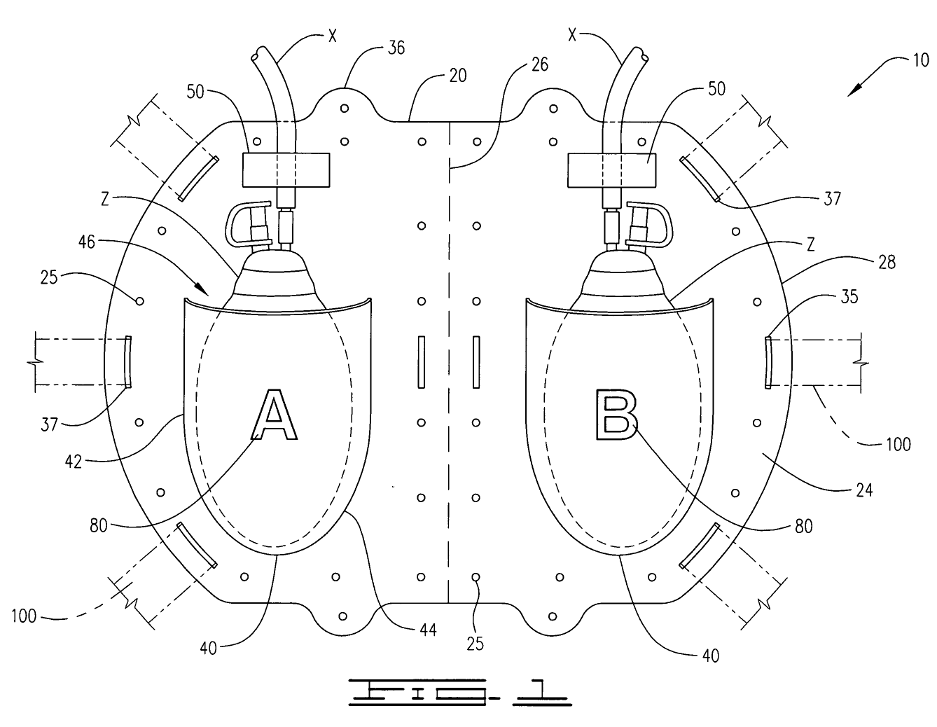

The universal application drain bulb and tubing support system 10, shown in FIGS. 1-8, comprises a flat base structure 20 defining a front surface 24 and rear surface 22, a perimeter 28, and a plurality of patient attachment sites 35, one or more defined pockets 40 attaching to the front surface 24 configured to secure and retain a respective medical drain bulb Z, a respective drain tubing anchor 50 positioned above each pocket 40 and attached to the front surface 24 of the flat base structure 20 and other optional elements as defined below.

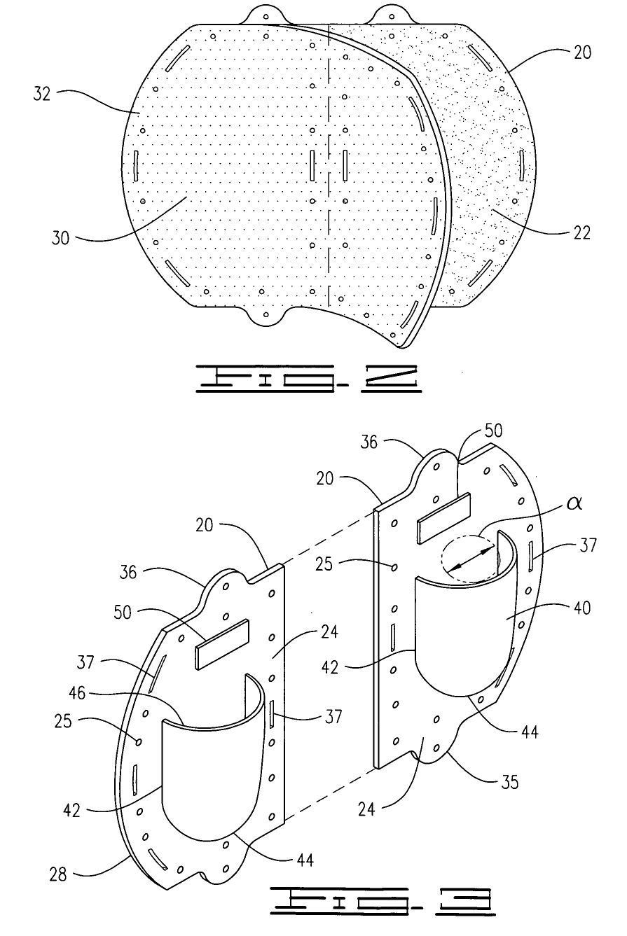

The flat base structure 20 is shaped in a manner to conform to a patient and multiple locations, depending upon the proximity to the drain tubing X installation location, FIG. 9. The base structure 20 should be flexible so that is has the ability to contour to the various potential locations of placement. The rear surface 22 also provides for the application of an skin-friendly adhesive 30 to attach the rear surface 22 directly to a patient, FIG. 2. Each attachment site 35 is integrated into the perimeter 28 of the flat base structure 20 and are selective based upon practical attachment using bands, belts, straps 100 or even adhesive tape, depending upon the election of the health care provider. For example, the flat base structure 20 may be hung around the neck, strapped to the chest, attached around the waist, around a leg or arm, on the chest or back, or anywhere the health care provider deems most appropriate, FIG. 9. Part of the fluid evacuation process involves a combination of suction and gravity, so depending on where the drain tubing X is located, placement of the flat base structure 20 and the manner of attachment would be broad and variable. Each patient attachment site 35 may define an extended tab 36, an integrated slot 37 from the front surface 24 through the rear surface 22, FIG. 1, a hole, other shaped flat extension element or a combination of such. Selected materials for the flat base structure 20 may include silicon, polymeric plastics or synthetic materials, foam laminate materials, fabric, rubber or other hypo-allergenic materials that are sanitized and able to be periodically cleaned or disposed of after use. The flat base structure 20 may be cut from a large sheet of material or molded into form or otherwise provided from the selected materials through known manufacturing processes.

Each pocket 40 is attached to the front surface 24 of the flat base structure 20 and projects outward from the front surface 24, further defining lateral sides 42 and a lower pocket floor 44 which conform each pocket 20 to accept a singular drain bulb Z from an upper opening 46, FIGS. 1 and 3. The lateral sides 42 and lower floor 44 define each pocket 40 with internal dimensions α conforming to the outer dimensions of the drain bulb Z being placed within each pocket 40, the pocket 40 applying sufficient pressure upon the drain bulb Z to secure it, but without applying a positive pressure on the drain bulb Z or cause compression.

Each universal application drain bulb and tubing support system 10 may provide more than one pocket 40 attached to the outer surface 24 of a common flat base structure 20, FIG. 1, providing for the securement of more than one drain bulb Z. One of the optional elements provided within the system may include a perforation 26, FIG. 1, between multiple pockets 40 on a common flat base structure 20 allowing the medical provider to cut the flat base structure 20 between pockets 40 as required to properly locate multiple drain bulbs Z. These perforations 26 may be cut with available medical equipment or simply separated by an applied force across the perforation 26, provided the flat base structure 20 is made from the appropriate materials that can be cut or separated, which is the preferred embodiment.

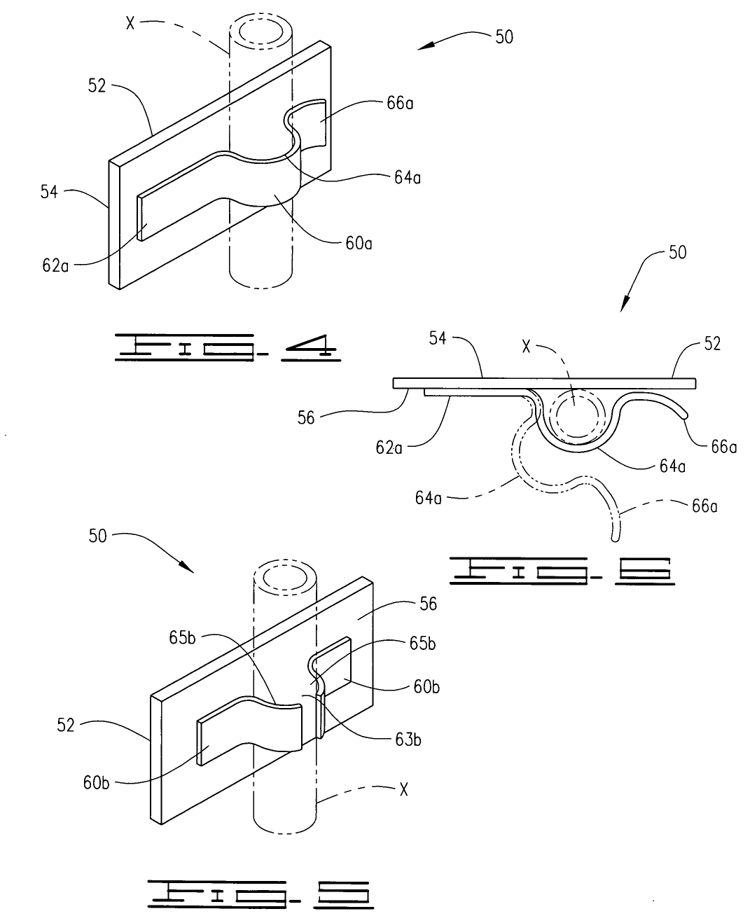

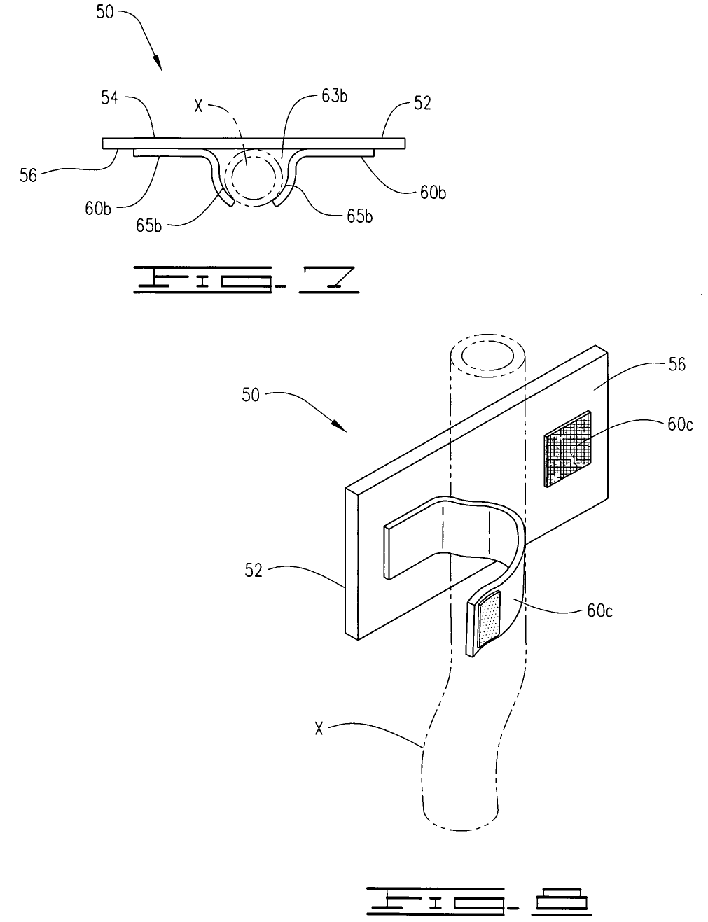

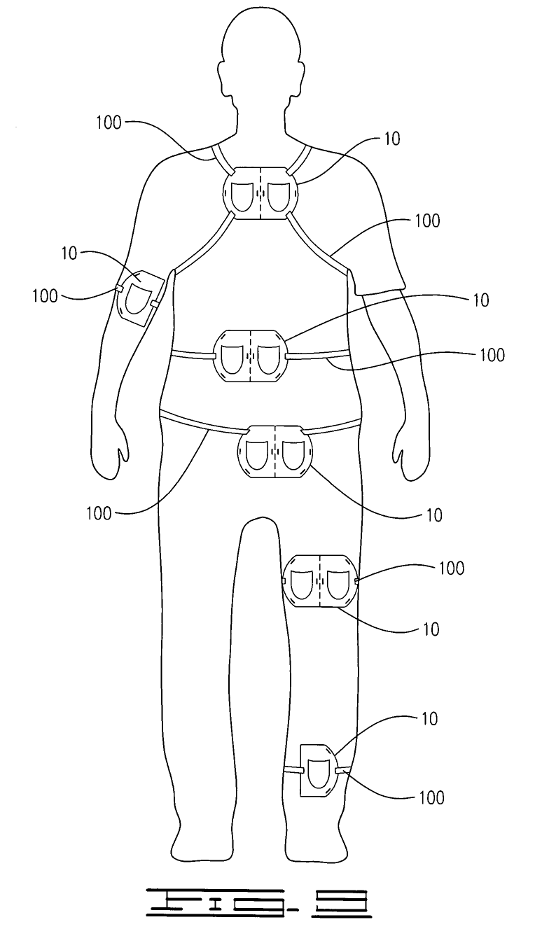

The tubing anchors 50 are an essential part of the system, FIGS. 1 and 4-8. The tubing anchors 50 are provided to secure each segment of drain tubing X exiting the drain bulb Z for additional security and to maintain the position and integrity of the drain bulb Z in proper orientation during storage within a respective pocket 40, FIG. 1. Each tubing anchor 50 possesses the utility characteristics of providing a quick, simple and secure means of installing and removing the drain tubing X, security to the flat base structure 20 to ensure proper alignment of each tubing anchor 50, and applying a force to the drain tubing X without restriction of fluid flow within the drain tubing X. To accomplish these utility characteristics, the tubing anchor 50 will define one or more embodiments. A first embodiment, FIGS. 4-5, provides an anchor base 52 having a rear surface 54 attached to the front surface 24 of the flat base structure 20 above a respective pocket 40 and central to the upper opening 46 of each pocket 40 by an adhesive or other means of integration. A front surface 56 of each first embodiment tubing anchor 50 provides a resilient extension arm 60a providing a first end 62a secured to the front surface 56 of the anchor base 52, a curved armature 64a and a second end 66a for inserting and securing the drain tubing X between the front surface 56 of the anchor base 52 and the curved armature 64a. In a second embodiment, FIGS. 6-7, the same anchor base 50 is provided, but a pair of resilient arms 60b defining an inner semi-circular margin 65b are attached to the front surface 56 of the anchor base 52, each semi-circular margin 65b directed towards each other forming a circular opening 63b between each pair of resilient arms 60b, the circular opening 63b having a diameter less than the diameter of the drain tubing X and receiving and securing the drain tubing X upon insertion within the circular opening 63b without restriction of the drain tubing X once installed. A third embodiment would provide the same anchor base with a single arm having a first end which is hinged or pivotally attached to the front surface of the anchor base, the single arm providing an elevated portion within which the drain tubing is positioned, with a second end of the single arm pivoted over the inserted drain tubing and locked in place by an arm locking means extending from the front surface of the anchor base to secure the single arm over the drain tubing until intentionally unlocked by a care giver or patient. Many other tubing anchors are contemplated within the scope of this system, including fabric hook and loop straps 60c, FIG. 8, ties, snap hooks, bottons and button holes, snaps, straps, ties, resilient metal structures, plastic molded structures and other tubing security means adapted to drain tubing X.

In versions of the drain bulb and tubing support systems 10, there may be additional features defined within the system which are contemplated as part of the system. Each pocket 40 in the system versions providing for a plurality of pockets 40 mounting to a common flat base structure 20, each pocket 40 may contain an icon, number or letter 80, FIG. 1, which identifies each pocket 40 and distinguishes one pocket 40 from another. This may be beneficial where the patient has multiple drain tubing X and drain bulbs Z at once, wherein the health care provider may readily identify the drain bulb Z contained within each pocket 40 by a recorded corresponding identification assignment to assist in proper assessment of the efficiency and effect of each drain bulb Z. These icons 80 also assist is identification of the corresponding drain tubing X where there is a problem due to entanglement, wound displacement and subsequent medical examination and observation. Another optional feature of the drain bulb and tubing support system 10 may include a peel-off backing 32, FIG. 2, applied to the dermal friendly adhesive 30 on the rear surface 22 of the flat base structure 20 which is optionally removed for direct application of the flat base structure 20 to the patient at a desired location or at multiple locations. The flat base plate 20 may also include small air holes 25 to provide some air flow through the flat base structure 20 to the patient, reinforcement materials laminated into the flat base structure 20, elastomeric materials integrated within the flat base structure 20 to allow for the flat base structure 20 to be stretched at intended locations within the flat base structure 20, as well as rigid panels to restrict stretching in other locations.

While the drain bulb and tubing support system 10 has been particularly shown and described with reference to a preferred embodiment thereof, it will be understood by those skilled in the art that changes in form and detail may be made therein without departing from the spirit and scope of the invention.

What is claimed is:

1. A universal application drain bulb and tubing support system for securing surgical drain tubing and drain bulbs subsequent to a surgical procedure, said system comprising:
a flat base structure defining a rear surface, a front surface, and a perimeter providing a plurality of attachment sites;
at least one pocket defining lateral sides, a pocket floor and an upper opening, said pocket configured to receive and secure a single surgical drain bulb without constriction to said surgical drain bulb;
a tubing anchor defining an anchor base with a rear surface attached to said flat base structure and centered above said upper opening of each of said at least one pocket and a front surface removably securing said surgical drain tubing attached to said surgical drain bulb to said tubing anchor, wherein said flat base structure is secured at a desired location to a post-surgical patient by a belt, a strap, or band to one or more attachment sites or an adhesive applied to said rear surface of flat base structure.

2. The universal application drain bulb and tubing support system of claim 1, each said attachment site defining an extension tab or slot.

3. The universal application drain bulb and tubing support system of claim 1, said flat base structure further defining a perforation to provide separation of said flat base structure between said pockets where said flat base structure provides for two or more pockets extending from said front surface.

4. The universal application drain bulb and tubing support system of claim 1, wherein said adhesive is a bio-compatible adhesive pre-applied to said rear surface with a peel-off backing to cover said adhesive until application.

5. The universal application drain bulb and tubing support system of claim 1, said tubing anchor further comprising:
said front surface defining a resilient extension arm having a first end secured to said front surface, a curved armature and a second end under which said surgical drain tubing is inserted, thereby securing said surgical drain tubing within said curved armature of said resilient extension arm.

6. The universal application drain bulb and tubing support system of claim 1, said tubing anchor further comprising:
said front surface providing a pair of resilient arms defining an inner semi-circular margin, each said semi-circular margin directed towards another forming a circular opening between each said pair of resilient arms, said circular opening having a diameter less than a diameter of said surgical drain tubing receiving and securing said surgical drain tubing upon insertion within said circular opening without restriction of said surgical drain tubing.

7. The universal application drain bulb and tubing support system of claim 1, said tubing anchor further comprising a hook and loop strap attaching said surgical tubing anchor to said tubing anchor.

8. The universal application drain bulb and tubing support system of claim 1 further defining any other element as shown or disclosed within the specification and drawings.
